(19) United States
(12) Patent Application Publication
(10) Pub. No.: US 2017/0251921 A1
(43) Pub. Date: Sep. 7, 2017

(54) OPTICAL INTRAOCULAR SENSOR AND SENSING METHOD

(71) Applicant: The Regents of the University of California, Oakland, CA (US)

(72) Inventors: Alex Phan, La Jolla, CA (US); Phuong Truong, La Jolla, CA (US); Robert N. Weinreb, La Jolla, CA (US); Juan Lasheras, La Jolla, CA (US); Frank Talke, La Jolla, CA (US)

(21) Appl. No.: 15/518,434

(22) PCT Filed: Oct. 20, 2015
(86) PCT No.: PCT/US15/56449

§ 371 Date: Apr. 11, 2017

Related U.S. Application Data
(60) Provisional application No. 62/065,982, filed on Oct. 20, 2014.

(51) Int. Cl. A61B 3/16; A61B 3/14

(52) U.S. Cl. CPC A61B 3/16; A61B 3/14

(57) ABSTRACT

An optical pressure sensor sized to be implanted at an intraocular location and formed from biocompatible materials. The sensor includes a rigid structure that supports a deformable structure arranged such that deformation of the deformable structure can be monitored optically when implanted in the intraocular location. A method for sensing intraocular pressure images the deformable structure and correlates an optical property affected by the state of deformation of the deformable structure to an intraocular pressure.

### PRIORITY CLAIM AND REFERENCE TO RELATED APPLICATION

The application claims priority under 35 U.S.C. §119 and all applicable statutes and treaties from prior provisional application Ser. No. 62/065,982, which was filed Oct. 20, 2014.

### FIELD

A field of the invention sensors and sensing, particularly ocular sensors and sensing.

### BACKGROUND

Ocular sensors and sensing are important to monitor patient intraocular pressure (IOP). Ocular tonometry techniques are currently used in standard practice to monitor IOP. These techniques provide only a snapshot of the pressure profile and give an indirect measurement of IOP.

More recently, there have been efforts to develop implantable sensors using MEMS (micro electromechanical systems) technology. Many of these devices use capacitive sensing and require electrical components including batteries. Difficulties with these devices include signal readout, size, sensitivity, power consumption and biocompatibility.

Active implants that include active components such as transducers, modulators, microprocessors and transmitters are disclosed in the following publications.

J. Coosemans, M. Catrysse, and R. Puers, “A readout circuit for an intraocular pressure sensor,” Sens. Actuators A, vol. 110, pp. 432-438, 2004.

R. Puers, “Linking sensors with telemetry: Impact on system design,” in Proc. 8th Int. Conf. Solid-State Sens. Actuators, Eurosens. IX, Stockholm, Sweden, Jun. 25-29, 1995, pp. 169-174.

K. Stangel, S. Kolnsberg, Hammerschmidt, H. K. Trieu, and W. Mokwa, “A programmable Intraocular CMOS pressure sensor system Implant,” IEEE J. Solid State, vol. 36, no. 7, pp. 1094-1100, July 2001.

W. Mokwa and U. Schnakenberg, “Micro-transponder systems for medical applications,” IEEE Trans. Instrument. Measure., vol. 50, no. 6, pp. 1551-1555, December 2001.

There are some prior passive IOP sensors. One system and method for sensing intraocular pressure is based on detecting spectrum shift in reflectance of a nano photonic structure to monitoring IOP. This method requires a specialized spectrometer to send in infrared light and obtain reflecting light which renders it inconvenient for users. Complex fabrication process of multiple layers of nanophotonic structure requires high precision and may lead to issue with reliability.

The following publications discuss passive implants where wireless monitoring of the IOP is achieved through mutual inductance coupling between the inductor on the sensor and the external loop antenna.

C. C. Collins, “Miniature passive pressure transensor for implanting in eye,” IEEE Trans. Bio-Med. Eng., vol. BME-14, no. 2, pp. 74-83, April 1967

Y. Backlund, L. Rosengren, B. Hok, and B. Svedbergh, “Passive silicon transensor intended for biomedical, remote pressure monitoring,” Sens. Actuators, vol. A21-A23, pp. 58-61, 1990.

L. Rosengren, Y. Backlund, T. Sjostrom, B. Hok, and B. Svedbergh, “A system for wireless intraocular pressure measurements using a silicon micromachined sensor,” J. Micromech. Microeng., vol. 2, pp. 202-204, 1992.

L. Rosengren, P. Rangsten, Y. Backlund, B. Hok, B. Svedbergh, and G. Selen, “A system for passive implantable pressure sensors,” Sens. Actuators A, vol. 43, pp. 55-58,1994.

K. Van Schuylenbergh and R. Pures, “Passive telemetry by harmonics detection,” in Proc. 18th Annu. Int. Conf. IEEE Eng. Med. Biol. Soc., Amsterdam, The Netherlands, 1996, vol. 1, pp. 299-300.

R. Puers, G. Vandevoorde, and D. De Bruyker, “Electrodeposited copper inductors for intraocular pressure telemetry,” J. Micromech. Microeng., vol. 10, pp. 124-129,2000.

O. Akar, T. Akin, and K. Najafi, “A wireless batch sealed absolute capacitive pressure sensor,” Sens. Actuator A, vol. 95, pp. 29-38,2001.

I. Araci, B. Su, S Quake, Y. Mandel, “An implantable microfluidic device for self-monitoring of intraocular pressure,” Nature Medicine 20,1074-1078, 2014.

Choo, Hyuck, David Sretavan, and Myung-Ki Kim. System and Method for Sensing Intraocular Pressure. Patent W02013090886 A1. 20 Jun. 2013.

Chen, Po-Jui, Damien Rodger, Mark Humayun, Yu-Chong Tai. “Unpowered spiral-tube parylene pressure sensor for intraocular pressure sensing.” Sensors and Actuators A: Physical, vol. 127, pp. 276-282, 2006.

Ghannad-Reizaie, M. “A powerless optical microsensor for monitoring intraocular pressure with keratoprostheses.” Solid-State Sensors, Conference. IEEE, 2013.

An implantable microfluidic device for self-monitoring of intraocular pressure has been implemented based on measuring the displacement of a gas-fluid interface as a function of pressure. This is described in Araci, et al., supra [0019]. This design suffers from difficulty with detecting the gas-fluid interface due to low contrast. One end of the channel is open to aqueous humor in the anterior chamber which makes it susceptible to clogging due to protein deposition. There is also the potential of gas leaking through the sensor walls over time compromising the device's integrity and reading accuracy.

A powerless optical microsensor for monitoring intraocular pressure with a keratoprostheses has been developed by Ghannad-Reizaie, M, supra [0022]. It is based on comparing relative reflectance intensities from two different layers of quantum dots in order to measure IOP. This complicated design poses difficulties during the manufacturing process along with high cost. It requires a specialized light source and detection unit to take measurement. Sensitivity is relatively low at 2 mmHg.

An unpowered spiral tube parylene pressure sensor for IOP sensing is based on detecting rotational displacement of the pointing tip of an Archimedean coil. The coil is open to the aqueous humor, which makes it susceptible to environmental changes. This could affect the device's sensitivity and reliability.

A contact lens with a microstraingauge embedded has been disclosed to measure changes in IOP by sensing the deformation of the corneal curvature by M. Leonardi, P. Leuenberger, D. Bertrand, A. Bertsch, and P. Renaud, “First steps toward noninvasive intraocular pressure monitoring with a sensing contact lens,” Investigative Ophthalmol. Vis. Sci., vol. 45, no. 9, Sep. 2004.

A technology that is state of the art in actual use and viewed favorably in the art is known as the Goldmann Applanation Tonometer. See, Kakaday, T, Hewitt AW, Voelcker NH, et al. “Advances in telemetric continuous intraocular pressure assessment.” British Journal of Ophthalmology., vol. 98, pp. 992-996, 2009. This technique and system measure IOP by applying a force that flattens the cornea. A plastic biprism contacts the cornea to provide an optical reference and optical viewing. The clinician adjusts pressure until optical reference semicircles come together as an indication of the IOP. This technique is conducted by doctors or clinicians, requiring close supervision. Some patients have trouble with this test, shying from the contact induced during the procedure. Some patients also tense, which can increase IOP during testing.

### SUMMARY OF THE INVENTION

An embodiment of the invention is an optical pressure sensor sized to be implanted at an intraocular location and formed from biocompatible materials. The sensor includes a rigid structure that supports a deformable structure arranged such that deformation of the deformable structure can be monitored optically when implanted in the intraocular location.

A preferred intraocular sensor includes a deformation structure arranged with respect to a rigid structure. Both are formed from or packaged within biocompatible materials and the sensor is sized to be installed at an intraocular location. The deformation structure deforms in response to intraocular pressures. The deformation structure is arranged to be imaged by an optical sensor when installed in the intraocular location such that deformation can be detected and measured. The deformation structure is preferably an elastomer materials. Example forms include columns and layers, periodic and irregular surfaces. Another preferred deformation structures include membranes and diaphragms. In a preferred embodiment, a membrane compresses one or more columns. In another embodiment, a diaphragm is suspended over a central cavity.

An optical intraocular sensor system includes an intraocular sensor of the invention. The system further includes a camera for sensing a characteristic of the deformation structure and a processor for correlating the characteristic to intraocular pressure by image analysis. In a preferred embodiment, the deformation structure is arranged to deform against the rigid structure and the processor correlates a contact area of the deformation structure against the rigid structure to intraocular pressure. In another preferred embodiment, the deformation structure is arranged to deform with respect to the rigid structure and the processor correlates a light intensity pattern to intraocular pressure. In another preferred embodiment, the deformation structure is arranged to deform with respect to the rigid structure and the processor correlates a light reflection pattern to intraocular pressure.

A preferred method of the invention senses intraocular pressure. The method includes implanting a sensor at an intraocular location. The sensor includes a rigid structure that supports a deformable structure. The sensor is subjected the sensor to light stimulation, imaging the deformable structure, and correlating an optical property affected by the state of deformation of the deformable structure to an intraocular pressure.

### BRIEF DESCRIPTION OF THE DRAWINGS

FIGS. 1A and 1B are schematic diagrams that illustrate a preferred embodiment sensor and sensor system of the invention;

FIGS. 2A and 2B are schematic diagrams that illustrate a preferred embodiment sensor and sensor system of the invention;

FIGS. 3A and 3B are schematic diagrams that illustrate a preferred embodiment sensor and sensor system of the invention;

FIG. 4 illustrates another preferred embodiment sensor and intraocular implantation locations;

FIGS. 5A and 5B are respectively an image and a schematic diagram that show an example macroscale experimental sensor device that was used to test sensing principles of the invention; FIGS. 5C and 5D show how the contact area increases with increasing pressure;

FIG. 6 illustrates a test set up used to obtain experimental data;

FIG. 7 is a data plot illustrating a linear relationship between applied pressure and contact area between an elastomer column and a rigid layer in an example experimental sensor device;

FIGS. 8A and 8B illustrate FEM typical deformation models in response to pressure loading;

FIG. 9 shows simulation results illustrating that contact area increased linearly with increasing applied pressure;

FIGS. 10A-10C show simulation results illustrating variation in membrane thickness for a preferred embodiment membrane sensor of the invention;

FIGS. 11A-11C include data concerning simulated changes to column height and the effect on normalized contact area;

FIGS. 12A-12C include data concerning simulated changes to column height and the effect on normalized contact area;

FIGS. 13A-13E illustrate a preferred embodiment fabrication process for a membrane sensor of the invention;

FIGS. 14A-14E illustrate another preferred embodiment fabrication process for a membrane sensor of the invention;

FIG. 15 illustrates data concerning the effect caused by different PDMS mixing ratios on flexibility of an elastomer column

FIG. 16A shows an additional preferred embodiment device that has been fabricated, and FIG. 16B is an image of a prototype of a device according to FIG. 16A;

FIGS. 16C and 16D illustrate a preferred sensing method of the invention;

FIGS. 17A-17F show an images of intensity bit maps taken from an experimental sensor at a fixed amount of elevated pressure and FIG. 17G is a plot of intensity change as a function of pressure change.

### DETAILED DESCRIPTION OF THE PREFERRED EMBODIMENTS

An embodiment of the invention is an intraocular sensor that offers the users the ability to monitor directly the IOP on a frequent basis using a wireless, passive, optically based pressure sensor. The sensor includes an elastomer that deforms in response to elevated intraocular pressures. A pressure amount and/or an IOP profile is calculated based on deformation of the elastomer, which includes deformable structures in preferred embodiments, e.g., columns, periodic or aperiodic structures, and membranes. An optical sensor device captures optical changes caused by the deformation, e.g. a change in the appearance and/or the light reflecting properties of the sensor due to pressure variation. Data are then analyzed to compute a pressure and/or pressure profile. A camera is an example optical sensor device.

In contrast to prior passive devices and sensing methods discussed in the background, a sensor device and method of the invention does not require specialized equipment such as a spectrometer, applanation tonometer, or detection unit. Data can be acquired and processed, for example, with a cell phone at the convenience of the user allowing a more accurate profile of IOP at arbitrary times. A pressure profile can be constructed based on changes in the appearance and/or the light reflecting properties of the sensor. The testing can be conducted under normal conditions without creating added tension to the subject being tested. Accordingly, some limitations of clinical Goldmann applanation tonomotery are avoided.

The present invention provides sensors that are biocompatible, passive, and sensitive. Preferred sensors of the invention are amenable to mass production at low cost using MEMS fabrication techniques.

A preferred embodiment sensor is an optically-based, passive, wireless intraocular pressure (IOP) sensor that detects small changes in pressure. An IOP profile calculation is based on deformation of an elastomer (e.g. columns, periodic structures, membranes, textured surfaces) in optical indication, e.g., appearance and/or light reflecting properties in response to the pressure changes. Preferred embodiment devices can be (1) integrated with an intraocular lens, (2) integrated with a glaucoma drainage device, (3) independently implanted at the surface of the iris, or (4) independently implanted to be free standing in the anterior chamber or capsule bag. Some of these options limit the surgical procedures necessary to install the sensor.

Preferred embodiment devices provide data acquisition and processing using a cell phone, tablet or other handheld computer device, or another computer device linked via wireless connection at the convenience of a user allowing accurate and frequent monitoring of IOP. In such embodiments, there is no need to use a specialized monitor, such as a spectrometer or detection unit. Sensors of the invention are biocompatible, and are passive. Sensors of the invention are readily fabricated using MEMS technology, and have a non-complex design and material structure. This permits low-cost manufacture. IOP data is readily obtained, transmitted and processed, locally or remotely.

Preferred devices have many ocular health monitoring applications. Patients that are at risk for glaucoma can be monitored and data can be used in a app on the cell phone or transmitted to a data center that performs analysis to identify conditions that trigger an alarm and raise flags that are transmitted to a care professional. In another application, IOP is monitored to establish target IOP for individual patient, and IOP data can be used to adjust intervention to achieve therapeutic goals.

Another important application is post-surgery monitoring. Ocular surgery patients such as cataract surgery patients are monitored after surgery to ensure that IOP remains in a healthy range.

Another application is as a research tool to aid and improve glaucoma studies or drug development in animal models. Other applications are those that require continuous tracking of changes in intraocular pressure such as during clinical trial.

Preferred devices provide data acquisition and processing using a cell phone at the convenience of the user allowing accurate and frequent monitoring of IOP. There is no need for specialized equipment such as a spectrometer or detection unit. Preferred embodiment devices provide IOP data to analyzed locally in a cell phone app or to be transmitted and processed, and incorporated into in-time-patient care remotely and wirelessly.

In a preferred embodiment, a present day smart phone camera is used to capture a optical indication of the deformation of an elastomer due to changes in pressure. The high resolution camera on many modern handheld devices can be used to capture deformation. Magnification lenses attached to the hand held device camera can aid detection. Magnifying lenses for cell phone and tablet cameras are commercially available. Of course, more standard optometric and clinical equipment can alternately be used to capture the deformation. An elastomer column is between two layers or an encasing structure, which receive intraocular forces. As pressure increases, the elastomer begins to deform between the encasing structure. In one embodiment, this deformation results in a change in the contact area, which is then used to calculate a pressure or pressure profile. In other embodiments, a change in the angle of reflected light or another optical indication of the deformation is used to calculate a pressure amount or pressure profile. In some embodiments, the encasing structure is preferably rigid, meaning that the encasing structure does not deform in response to intraocular pressures and will compress the elastomer column. In other embodiments, a membrane deflects to cause an optical change that can be measured.

Preferred embodiments of the invention will now be discussed with respect to the drawings. The drawings may include schematic representations, which will be understood by artisans in view of the general knowledge in the art and the description that follows. Features may be exaggerated in the drawings for emphasis, and features may not be to scale.

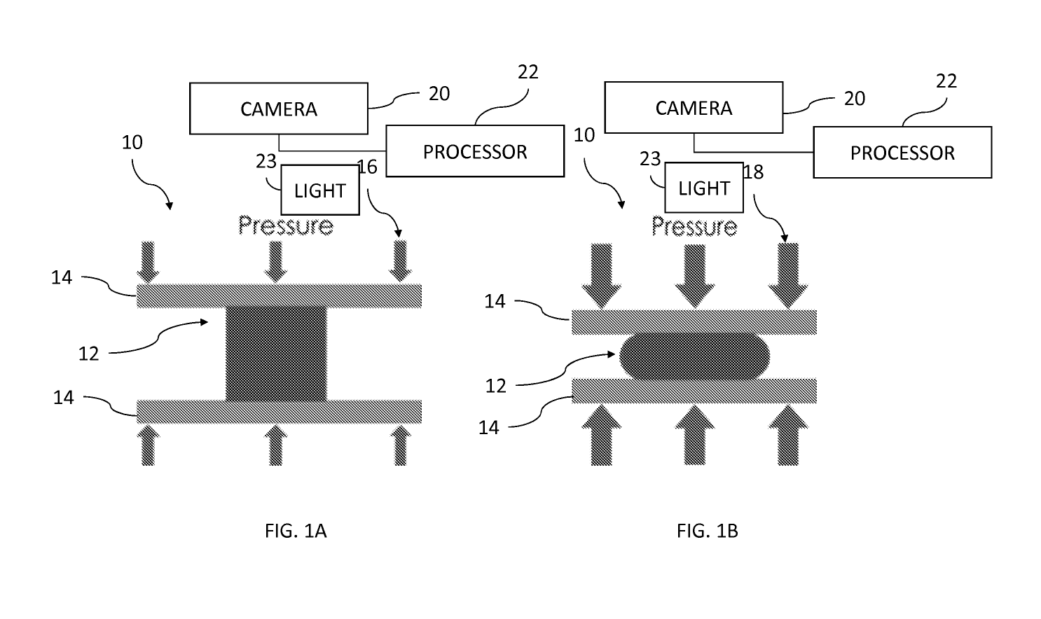

FIGS. 1A and 1B illustrate a preferred embodiment sensor 10 that is sized to be implanted in an eye. An elastomer structure 12, in the form of a column, is between two rigid layers or structures 14. When implanted, the layers or structures 14 are disposed to receive intraocular forces, and at least one is preferably transparent to measure change in contact area with the elastomer structure 12. FIG. 1A shows normal intraocular forces 16 characteristic of a healthy IOP. FIG. 1B shows elevated intraocular forces 18 characteristic of an IOP that would warrant medical attention. For simplicity of illustration, walls are not shown in FIGS. 1A and 1B, but wall enclose the sensor being bonded between the top and bottom layers or structures 14 and are flexible to compress to allow the layers or structures 14 to compress the elastomer 12. At a predetermined level exceeding a healthy IOP, the elastomer structure 12 begins to deform between the encasing layers or structures. This deformation results in a change in the contact area, which can then be used to calculate the pressure profile. The encasing layers or structures 14 are preferably rigid, meaning that the layers don't deform in response to normal intraocular pressures as shown in FIG. 1A and will compress the elastomer structure 12. The deformation is detected, for example, using a camera 20, which forms part of an IOP detection system. The camera 20 captures deformation of the elastomer structure 12, and a processor 22 can calculate pressure from a measured deformation. A light source 23 can stimulate a response.

The processing to correlate IOP to a reaction of the sensor 10 conducted by the processor 22 can be based upon various optical properties that change due to the compression of the elastomer structure 12, or the deflection of membranes and other elastomer features in additional embodiments. The response to pressure can change a focal point measured by the camera 20. It can also change the light intensity, reflected light wavelength, contact area, etc. These changed properties can be correlated to IOP, and determine a level of IOP.

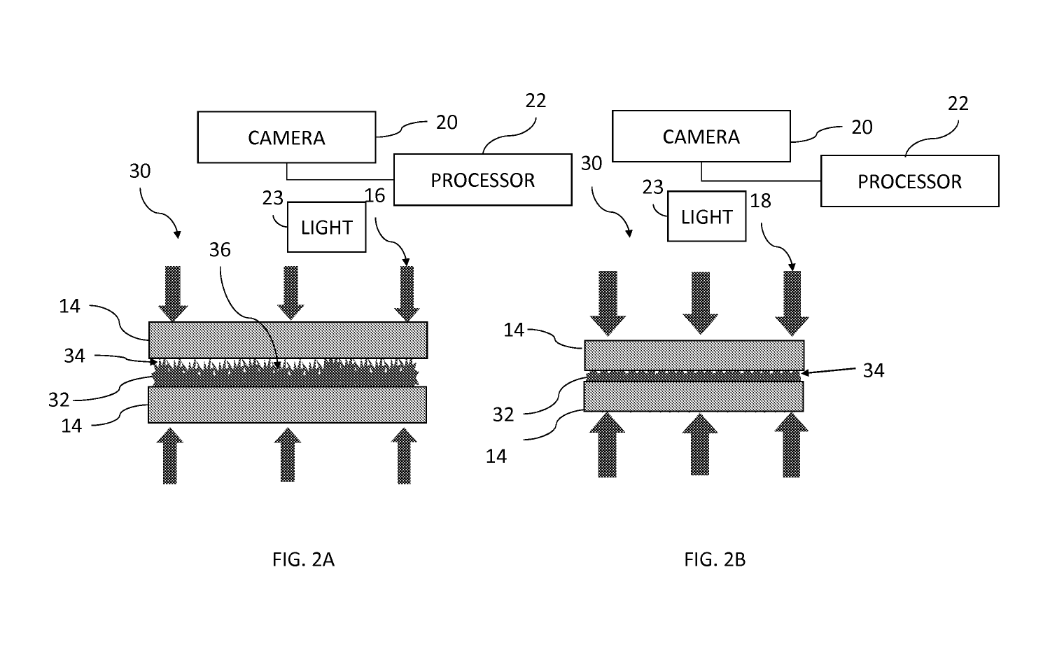

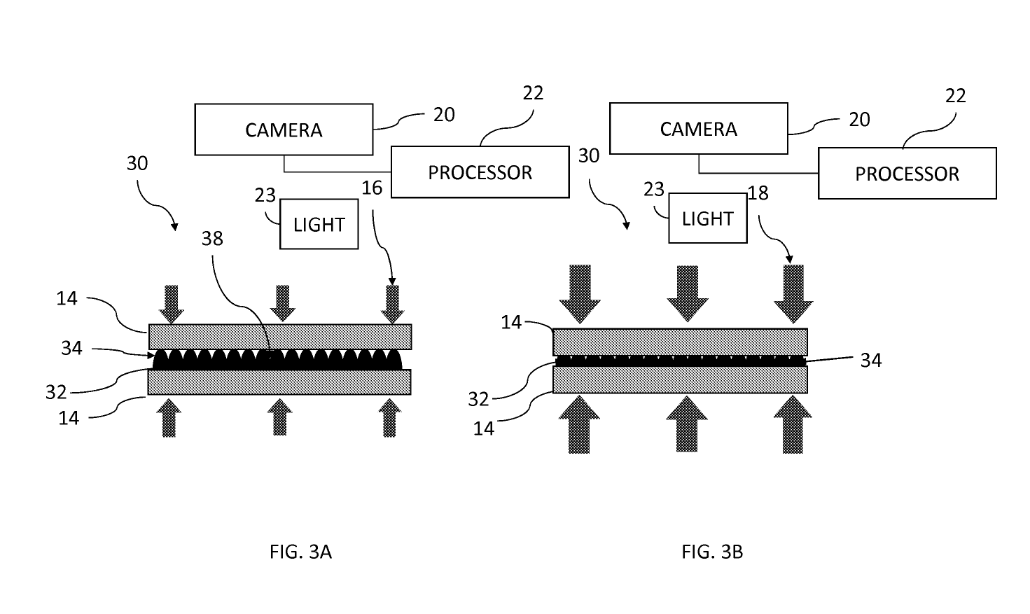

FIGS. 2A and 2B illustrate another preferred embodiment sensor 30. Instead of the elastomer structure 12 of FIGS. 1A and 1B, the sensor 30 includes a textured surface 32. The textured surface 32 is also formed of an elastomer material, but may includes a complex micro structured surface 34 as a basis for detection in IOP. The surface 34 gets compressed and light directed at the interface between parts 14 is attenuated depending on the surface roughness that changes in response to spacing between the two adjacent surfaces. 14 This provides for a detection mechanism when it deforms under elevated IOP as shown in FIG. 2B. The surface 34 deforms in FIG. 2B due to the variation of the external pressure to change the surface roughness and thereby the contact area of the textured surface layer 32 layer as pressure varies. The surface 34 is preferably an irregular surface, and the surface roughness changes with increasing pressure. In a particular preferred example embodiment, the surface 34 is configured as a rough, irregular surface with asperities 36 and increasing pressure results in compression of the asperities by the encasing layers or structures 14 and increases surface roughness contact with one or both of the encasing layers or structures 14. In other embodiments, the surface 34 forms a regular periodic structure on one or both surfaces of the elastomer layer 32 and the periodic structure changes with increasing pressure. FIGS. 3A and 3B illustrate such an embodiment, where the surface 34 includes periodic structures 38, e.g. rounded pillars, and increasing pressure results in compression of the periodic structures. Increasing pressure increases the contact area between the elastomer layer and the top layer or structure 14, as in the FIGS. 1A-2C embodiments. This provides a simple optical measurement to determine the change in the contact area that can be correlated to an IOP.

For any of the FIGS. 1A-3C embodiments, the elastomer material that deforms is sized, geometrically configured and selected from a sufficiently compliant material to deform in response to elevated IOP pressure. An example preferred elastomer material is PDMS with a Young's modulus of 360-800 KPa, and a Poison ratio of approximately 0.5. The material properties will depend upon the configuration of the sensor, and can be altered to obtain desired sensitivity. The sensor preferably has a sensitivity of at least 1 mmHg, meaning that a pressure change of 1 mmHg or more should induce a measurable deformation. Pressure changes are considered more important than force. The change in force is easily calculated by multiplying the change in pressure with the surface area of the region of interest. Other options include micro structured springs, half sphere structures, and other structures that will deform under typical elevated intraocular pressures. 2D micro spring and half sphere structures are feasible to fabricate on the correct scale from elastomer or other elastic materials. Another preferred embodiment includes an array of micro pillars instead of a single column.

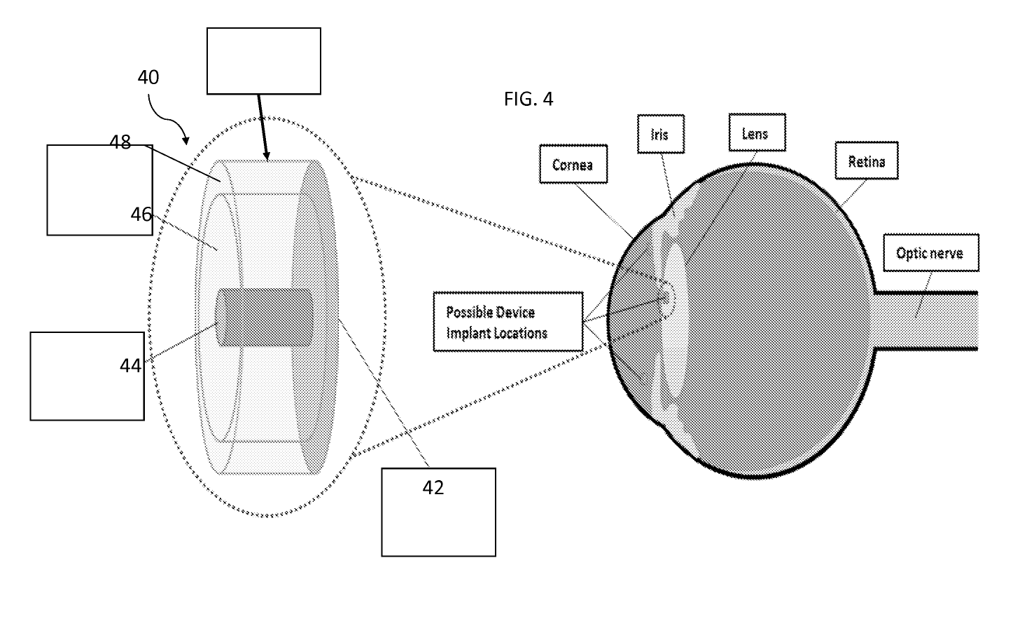

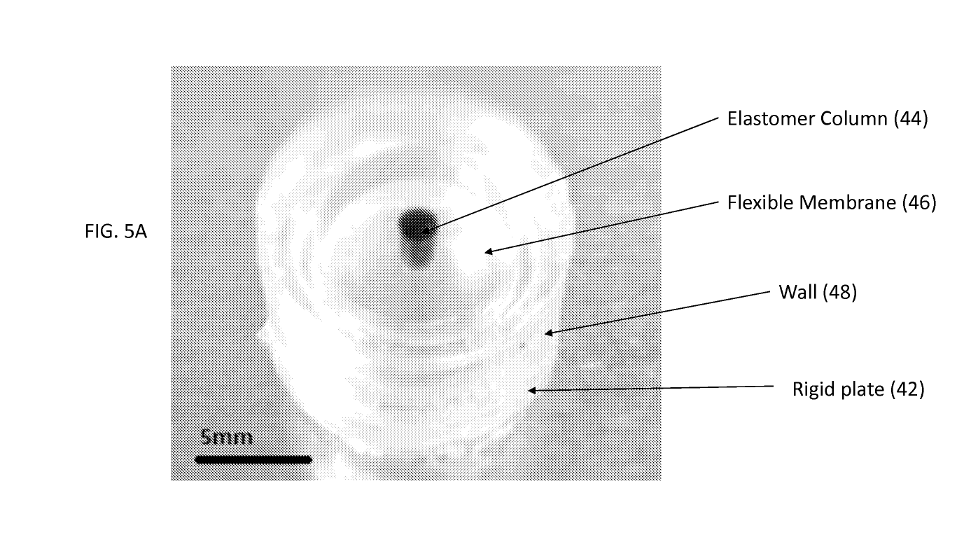

Another preferred embodiment sensor 40 consistent with the above illustrated embodiments is shown in FIG. 4 along with an illustration of locations for intraocular implantation. The example illustrated locations include the surface of the iris, free standing in the anterior chamber, capsule bag, integrated with intraocular lens, and integrated with a glaucoma drainage device. These are locations for any of the preferred embodiment sensors, which are sized to be surgically implanted and avoid interference with functioning of the eye.

The sensor 40 includes a rigid plate 42 and a column 44 placed between the rigid plate 42 and a membrane 46. A wall 48 seals the sensor. In FIG. 4, increases in IOP cause the membrane 46 to deflect downward which in turn compresses down on the column 44. Column deformation can be captured and used to calculate the IOP profile.

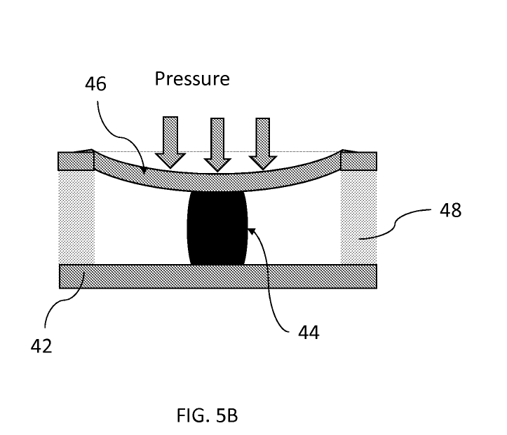

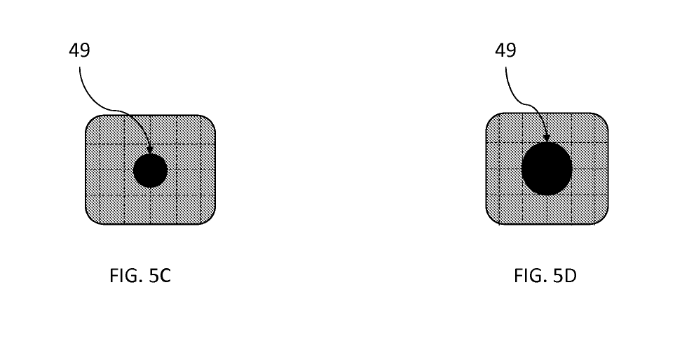

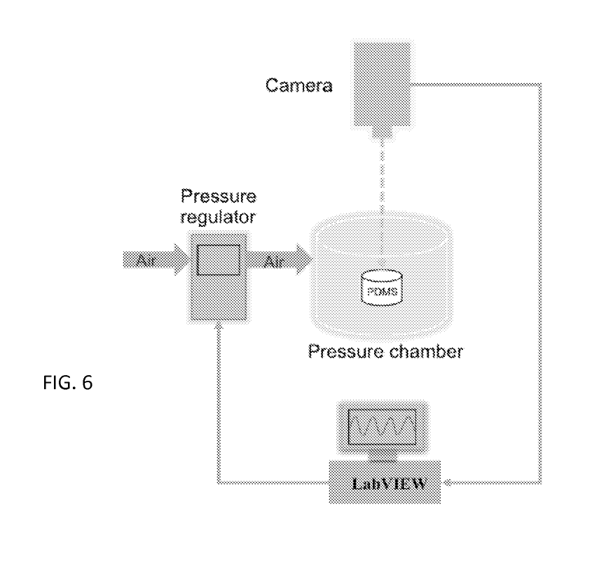

Experiments were conducted to test prototypes. The example sensors were fabricated consistently with FIG. 4 and used to demonstrate sensor parameters. FIGS. 5A shows an image of the example experimental device, which included a flexible PDMS membrane on top, a rigid glass bottom plate, a flexible PDMS column and PDMS walls sealing the top and bottom layers. FIG. 5B shows a schematic cross-sectional diagram of the experimental sensor under a condition of elevated pressure with the membrane 46 deflected to compress the column 44. FIGS. 5C and 5D show how a cross-sectional area 49 of the column 44 increases with increasing pressure, which is an optical property that can be measured over a range of different contact areas to create an IOP profile. If the contact area or the maximum diameter or foot print of this cross-sectional area is determined optically, a diameter or area ratio of deformed to undeformed column can be determined over a range of different pressures to create an IOP profile. The maximum diameter of the column 44 can be measured, for example, by determining the optical projection of column 44 on the bottom surface. Experimental results showed a linear relationship between foot print area and applied pressure. A sensitivity of 1 mmHg has been measured. The experimental set up to test the prototype is shown in FIG. 6.

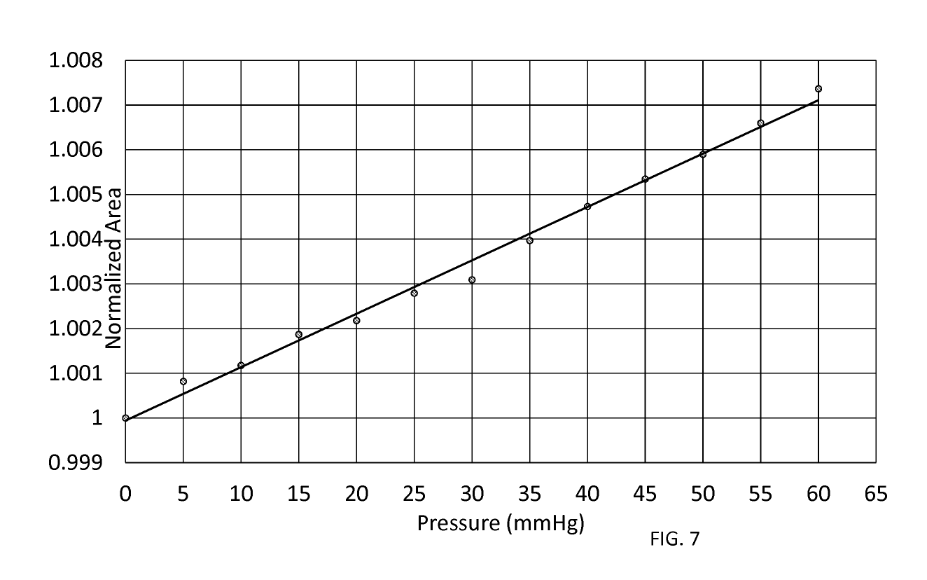

The experimental set-up was created to control the applied pressure, capture the contact area and plot the pressure profile. A schematic of the set-up is shown below in FIG. 6. In the testing, the sensor is placed inside the pressure chamber, a pressure regulator is used to control pressure inside the chamber, and a camera is mounted over the top of the sample. As the pressure is regulated up and down, the camera captures changes in contact area of the column and the membrane. Images are then analyzed at real time to calculate percentage change in contact area of the elastomer column against the membrane. The experimental results indicated a linear relationship between contact area and applied pressure, as indicated by the data in FIG. 7.

Miniaturized prototypes have also been made and used to explore the process of fabrication. These prototypes were consistent with FIG. 5A and 5B and had dimensions on the order of one millimeter, i.e. a diameter and height of approximately 1 mm.

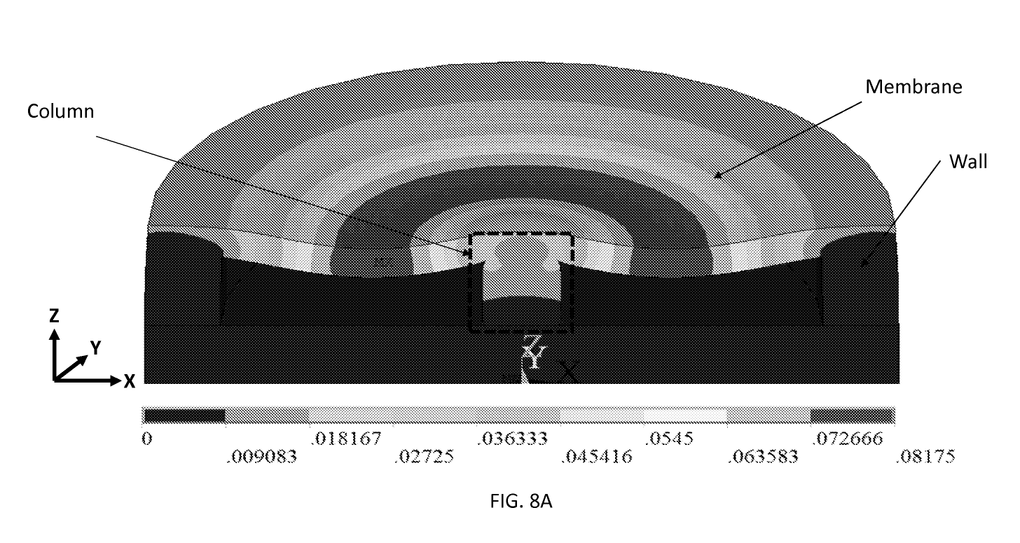

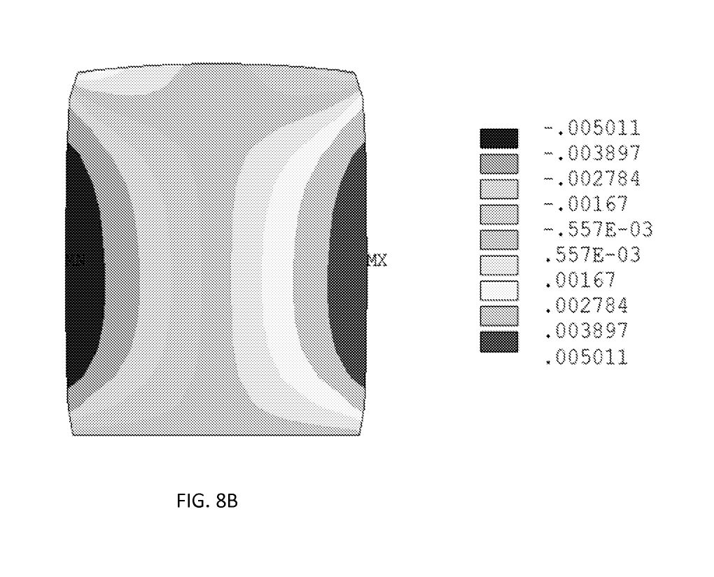

Finite element models were also built to simulate responses of the prototype under applied pressures. FIGS. 8A and 8B illustrate typical deformation models in response to pressure loading. FIG. 8A shows the overall deformation of the membrane.

FIG. 8B shows a simulation of the deformation of a typical column under transverse column loading. Maximum transverse deformation of the central column was determined. Results were then used to optimize designed parameters. Effects of the following parameters on maximum column diameter or cross sectional column area were investigated and are discussed below: pressure loading, membrane thickness, column height, and column width. In the simulation, the following parameters were used (except where varied as indicated below to test the effect caused by an individual parameter change. Column radius: 150 μm, column height: 300 μm, wall thickness: 200 μm, overall radius: 1000 μm, membrane thickness: 200 μm.

Effect of Pressure Loading

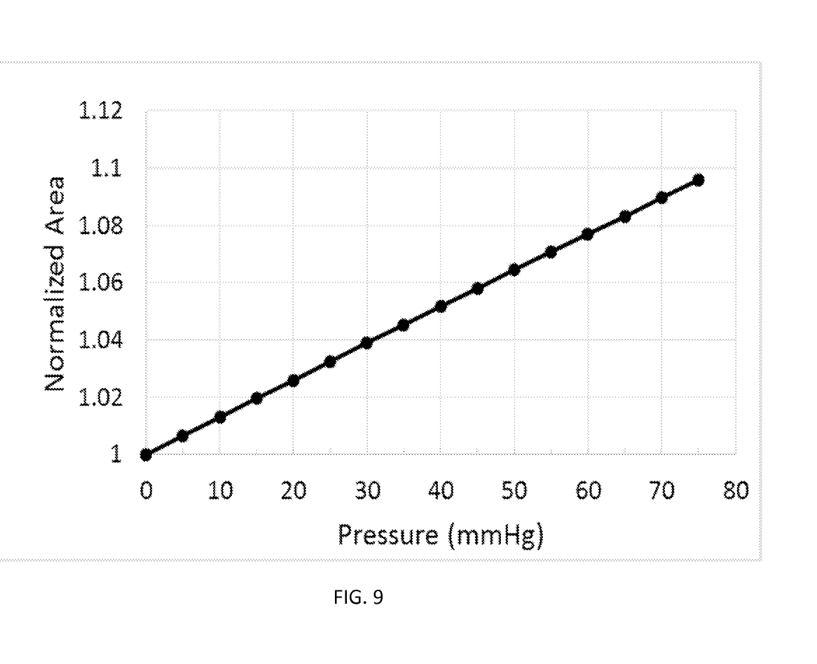

FIG. 9 shows simulation results illustrating that the diameter and column area at the contact increased linearly with increasing applied pressure. This simulated relationship correlates well with findings obtained from physical testing of macro scale prototype devices.

Effect of Membrane Thickness

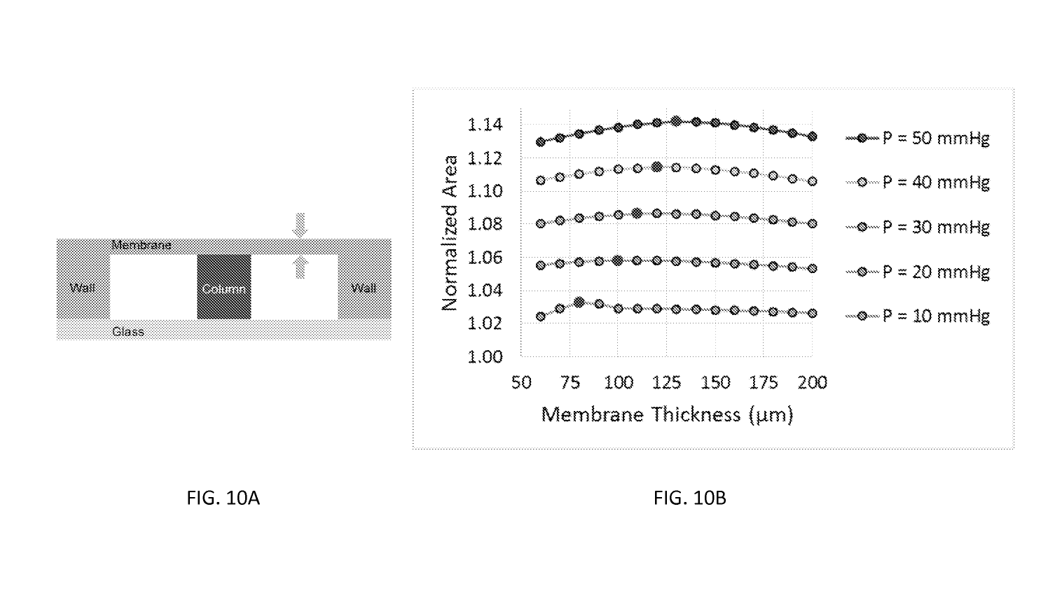

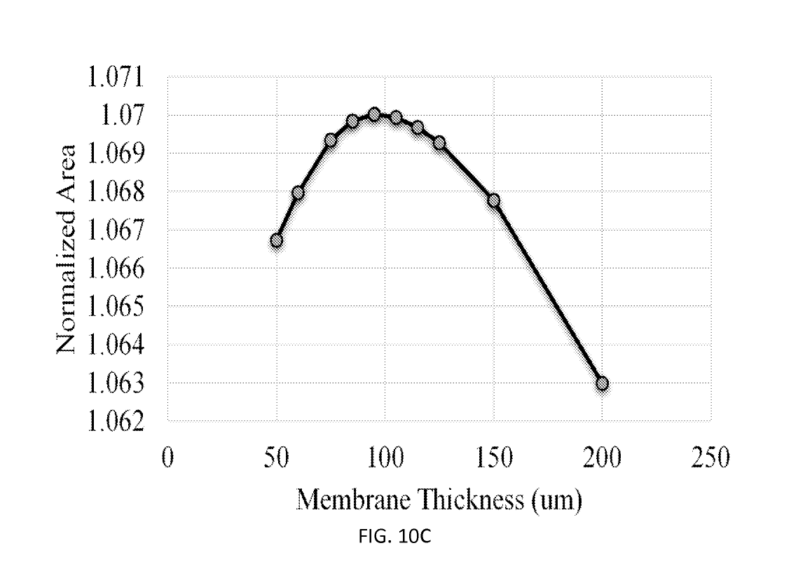

FIGS. 10A-10C shows simulation results illustrating variation in membrane thickness, which showed that the column cross-sectional contact area can be observed at an optimal membrane thickness. The effect of membrane thickness on the column cross-sectional contact area was studied, with the following parameters fixed: column radius of 125 μm, column height of 300 μm, wall thickness of 200 μm, overall radius of 1000 μm and applied pressure of 10 mmHg, 20 mmHg, 30 mmHg, 40 mmHg and 50 mmHg, while the membrane thickness was varied from 50 μm to 350 μm. These results indicate that there is an optimal membrane thickness for a given set of design parameters. This is due to the behavior of the membrane as it varies with thickness; namely, when the pressure is 20 mmHg and the membrane thickness is set above or below 95 μIn, there is a reduction in load acting on the column caused by the membrane's ability to withstand higher pressure. This leads to a smaller contact area than the optimal case. For a given set of materials and dimensions, an artisan can follow the FIG. 10 example and determine optimal membrane thickness by numerical simulation. The experiments varied one design parameter and fixed all others. This was done to determine which parameter is more sensitive to sensor performance. Simultaneous variation of multiple parameters can be used to determine an optimal set of design parameters.

Effect of Column Height

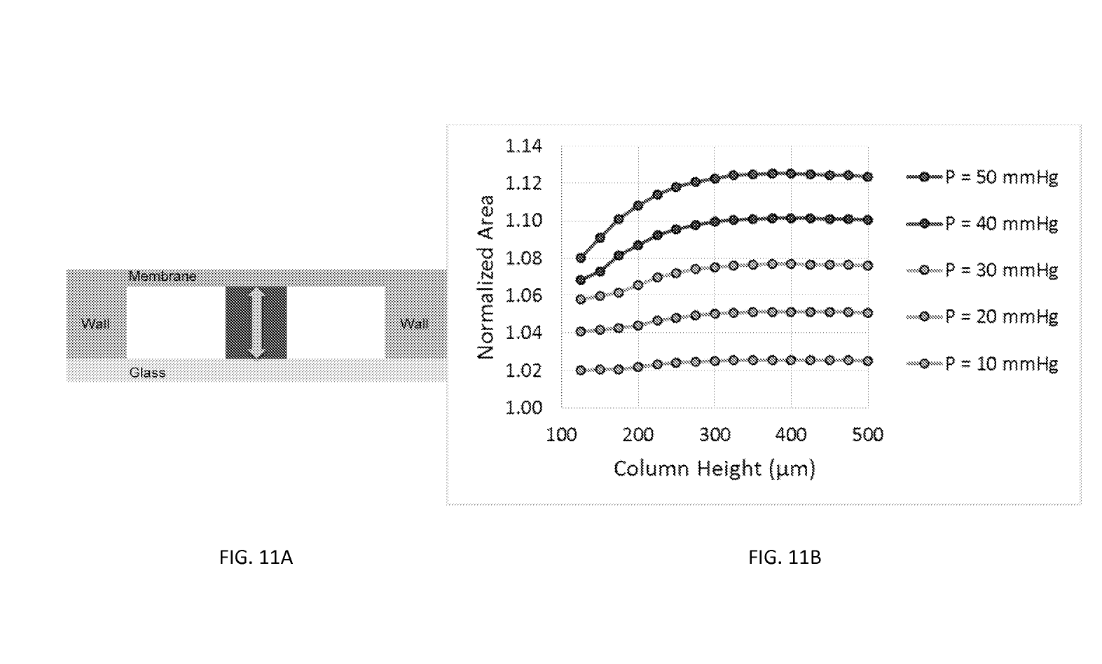

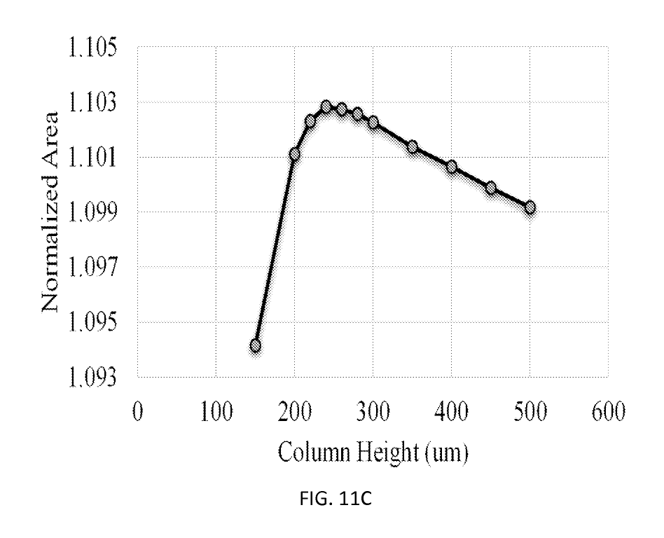

FIGS. 11A-11C include data concerning simulated changes to column height and the effect on normalized contact area. This data show a the column cross-sectional contact area with column height of 240 μm. For this simulation, the following parameters were fixed: column radius of 100 μm, membrane thickness of 100 μm, wall thickness of 200 μm, overall radius of 1000 μm and applied pressure of 10 mmHg to 50 mmHg, while column height was varied from 150 μm to 500 μm. This indicates that there is an optimal column height to achieve maximum contact area for a given set of parameters. When the column is too short, restrictions at the two fixed ends hinder the transverse displacement of the column, hence reducing the contact area. In example experimental devices, the minimum preferable column height was approximately 200 μm.

Effect of Column Width

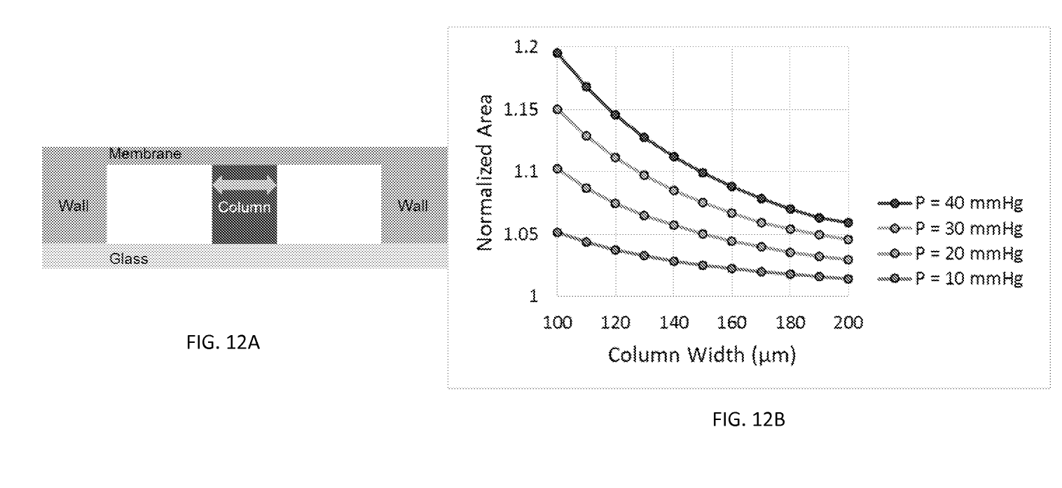

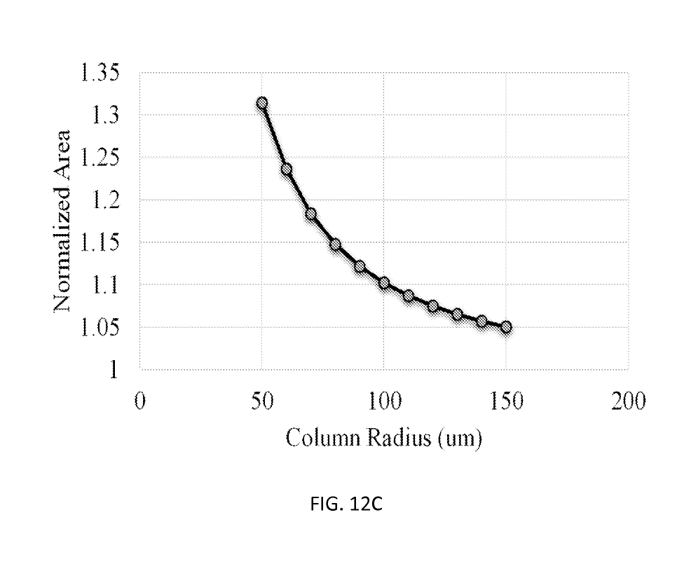

FIGS. 12A-12C include data concerning simulated changes to column height and the effect on normalized contact area. These data show an exponential increase in contact area as the column width decreases. The following parameters were fixed: column height of 240 um, membrane thickness of 100 um, wall thickness of 200 um, overall radius of 1000 um and applied pressure of 10 mmHg to 50 mmHg, while column radius was varied from 50 um to 150 um. Since membrane dimensions and pressure were kept constant, the amount of force exerted on the column also remains constant. Thus, with constant force, the reduction in column cross-sectional area leads to higher stress and larger transverse deformation. In general, a smaller column radius provides a larger normalized contact area deformation.

Summary of Simulation Experiments

The results and simulations showed that various embodiments can provide a linear and measureable contact area change in response to pressure. Measurement sensitivity of 1 mmHG was demonstrated over a range of 0 to 50 mm HG.

Preferred Fabrication Process

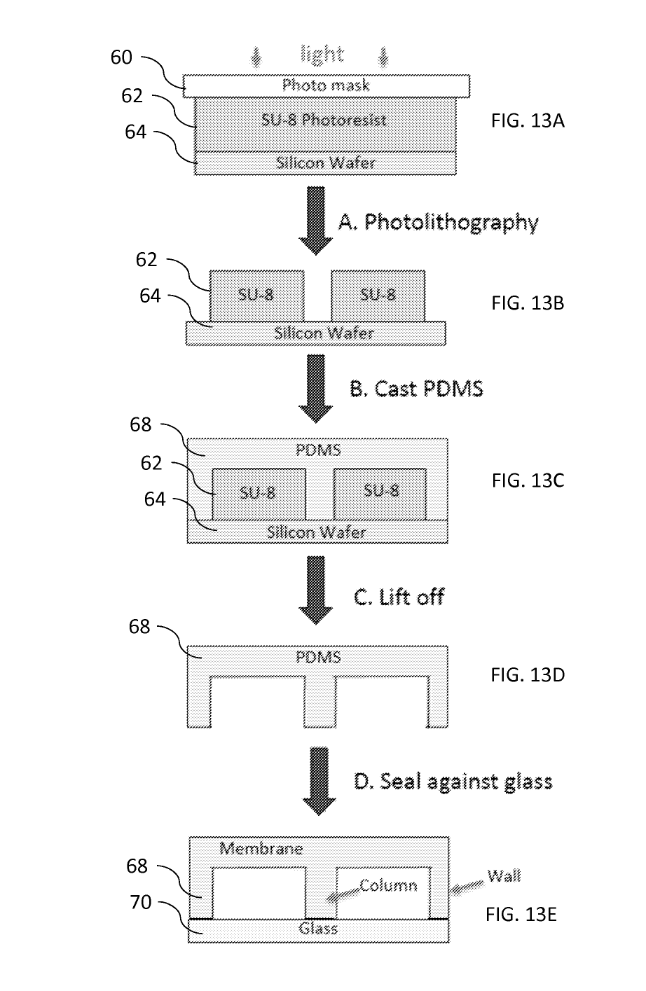

The simulated sensor of FIG. 8A that is consistent with FIG. 4 and FIG. 5 can be fabricated for implantation size via a preferred MEMs fabrication process that is illustrated in FIGS. 13A-13E. In FIG. 13A, a photomask 60 used to selectively expose photoresist 62, e.g. SU-8, on a semiconductor wafer 64, such as a silicon wafer. In FIG. 13B the lithography process is completed according to the pattern established by the photo mask. Elastomer material, e.g., PDMS, is deposited over the established pattern in FIG. 13C. A lift off releases the elastomer material, which is shaped as the membrane, column, and walls in FIG. 13D. This formed, unitary membrane structure 68 is then bonded to a transparent layer 70, such as a glass layer, in FIG. 13E. The wall thickness is controlled to make it effective stiff under the relevant pressure range 0-60 mmHg.

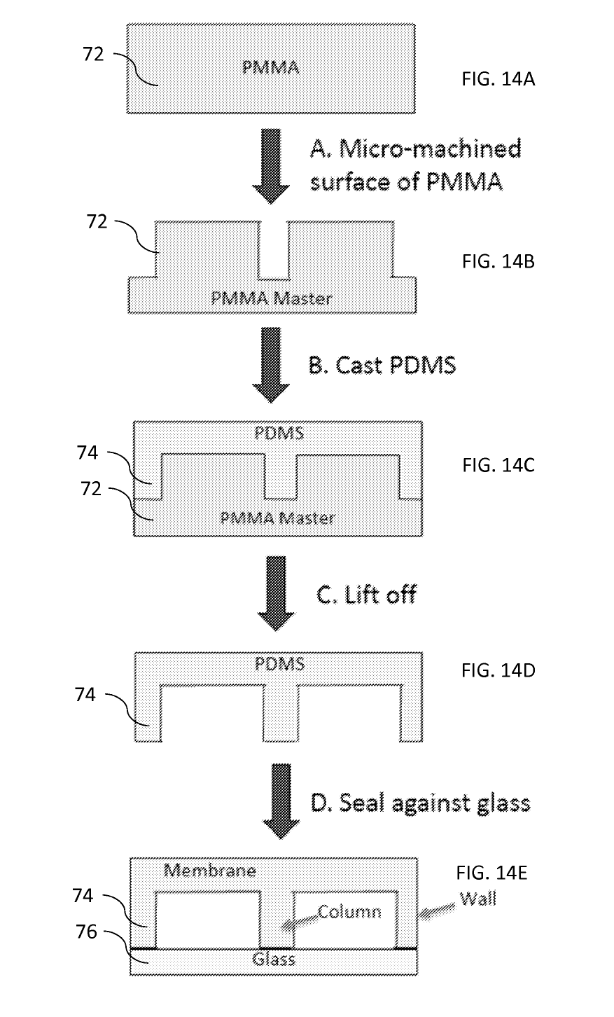

FIGS. 14A-14E show an alternate fabrication process that is a replica molding process. A mold material 14 in FIG. 14A, such as PMMA, is patterned by micro machining or by photo lithography in FIG. 14B. PDMS molding using photolithography can produce small features with high accuracy. Using micro CNC machining to create a master mold is an alternative. This method can produce larger features though some accuracy will be compromised. This completes formation of the master mold, and elastomer 74 is deposited and cured in FIG. 14C to form the unitary membrane and wall structure. This formed elastomer structure is released in FIG. 14D. It is challenging to make column heights larger than 200 μm using this technique.

Experiments using the FIG. 13 or 14 processes produced a membrane sensor having millimeter dimensions, specifically 3 mm diameter, 1 mm height, with a column that was 0.3 mm in diameter and 0.6 mm in height.

Material Effects

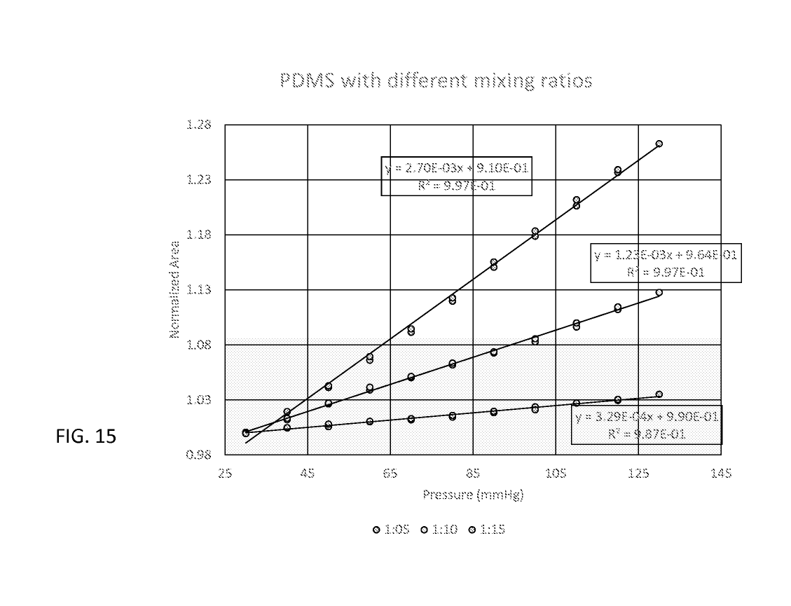

The thickness of the materials affects flexibility as discussed above. The particular materials selected, as well as the ratios of components of the materials can also affect the flexibility. Tests were conducted with example PDMS material of the column have mixing ratios of cross-linker to base polymer of 1:05, 1:10, 1:15 and the response to pressure is shown in FIG. 15. This data was obtained by applying pressure directly onto the column and capturing expansion of the column mid-section. The lower ratio is favorable for a more measurable response. All of the mixtures showed consistent response over 1000 cycles, with less than 5% variation. This will allow a sensor of the invention to provide results over a long period of time.

Additional Prototypes and Testing

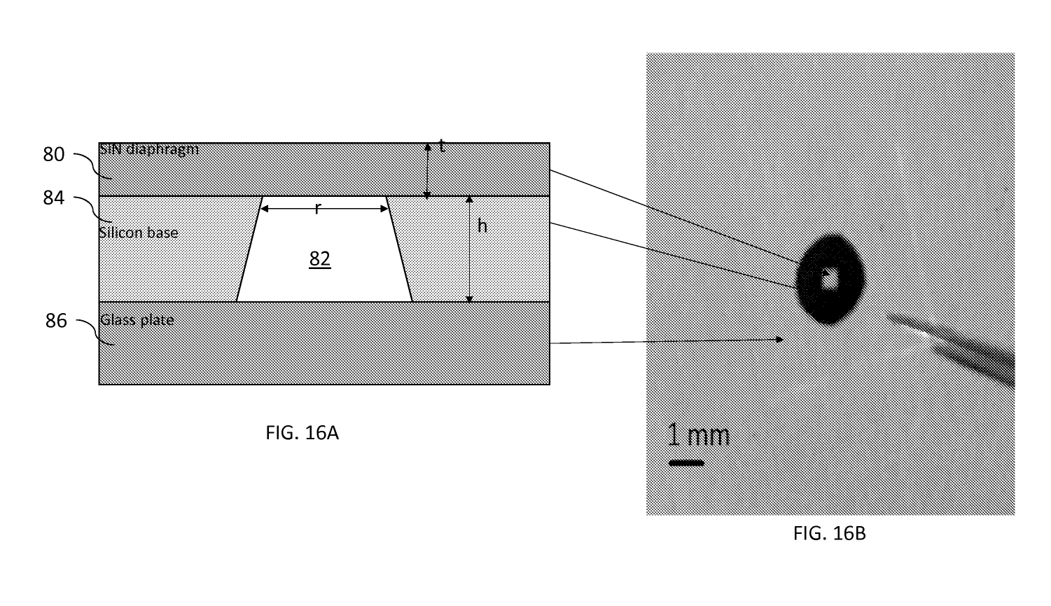

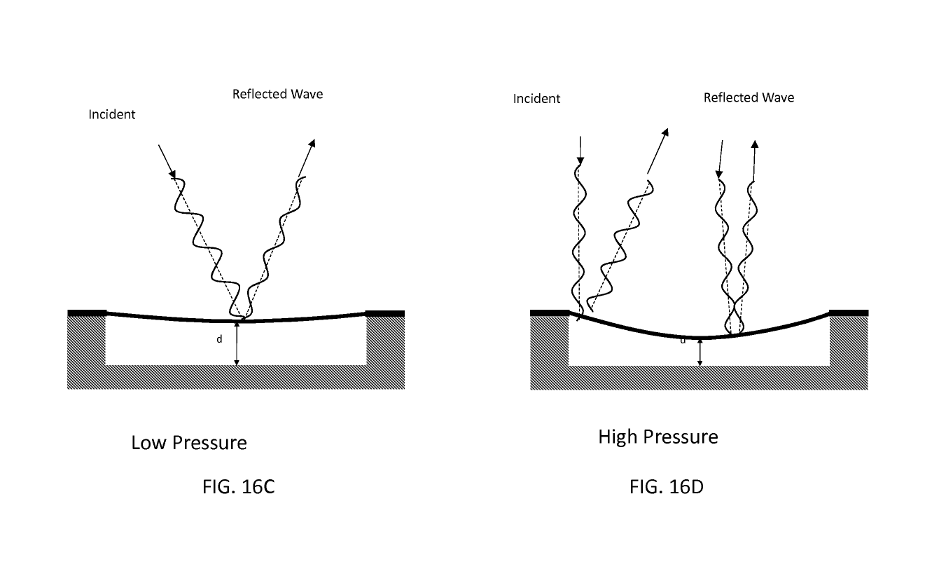

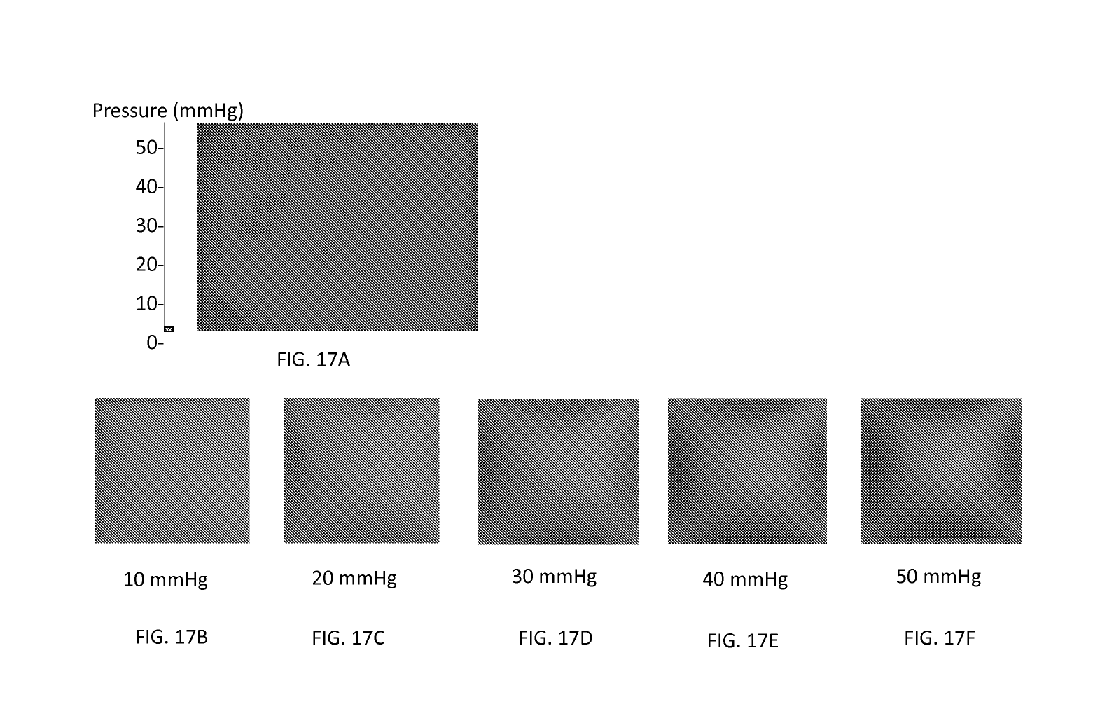

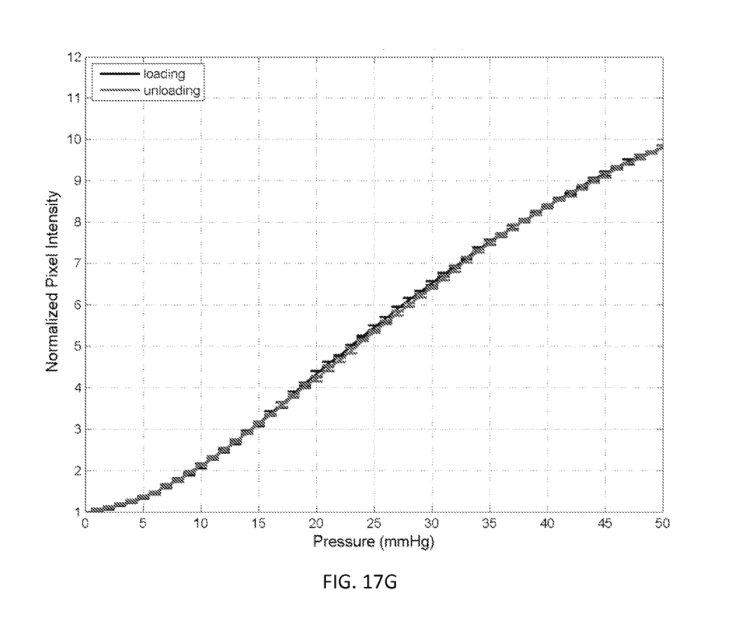

FIG. 16A shows an additional preferred embodiment device that has been fabricated, and FIG. 16B is an image of a prototype of a device according to FIG. 16A. In FIG. 16A, a flexible diaphragm layer 80, e.g. SiN, is suspended over a central empty volume 82 defined in silicon base 84 that is bonded to a glass plate, which could be another rigid material such as silicon. An alternate preferred sensing method that can be used with this embodiment (and other embodiments) is illustrated in FIGS. 16C and 16D, and is based upon the angle of light reflection change as the diaphragm 80 deflects an incident wave. Light reflection patterns can be correlated to specific intraocular pressures. The volume shape (and diaphragm portion that deflects) can be formed into a variety of shapes, cylinders, asymmetric polygons. Using a birefringent material (e.g., polystyrene, polycarbonate) for the diagphram enhances the light reflection contrast. Adding a lens on top of the diaphragm can further increase the contrast of the light reflection pattern. By fabricating a sensor with a cavity height d on the order of a few micro-meters, fringes can be obtained as in conventional interferometry, allowing calibration of the pressure using multi/mono-chromatic light interferometry. The image analysis can be improved in resolution by analyzing certain areas of the diaphragm in addition to analyzing the complete diaphragm. For example, the analysis can analyze the whole area or just the corners or just the diagonal lines. Fabricating sensors with various shapes ranging from polygon diaphragms to a circular diaphragm can yield higher resolution using this same measurement principle. FIG. 17A shows an image that is a bit map taken from an experimental sensor with pressure varied from 0 mmHg to 50 mmHg with step size of 1 mmHg, and FIGS. 17B-17F illustrate the bit maps at a serious of pressures. The prototype dimensions were: h=200 μm, t=50 nm, r=500 μm. As the pressure varies from 0-50 mmHg, the reflection pattern changes. Analyzing pixel intensity of these reflection patterns permits calculation of the applied pressure. FIG. 17G plots data of pixel intensity of reflection patterns against applied pressure. As pressure increases, the pixel intensity increases, which provides another.

While specific embodiments of the present invention have been shown and described, it should be understood that other modifications, substitutions and alternatives are to one of ordinary skill in the art. Such modifications, substitutions and alternatives can be made without departing from the spirit and scope of the invention.

Various features of the invention are set forth in the appended claims.

1. An optical intraocular sensor, comprising a deformation structure arranged with respect to a rigid structure, the deformation structure and rigid structure being formed from or packaged within biocompatible materials and being sized to be installed at an intraocular location, wherein the deformation structure deforms in response to intraocular pressures, and wherein the deformation structure is arranged to be imaged by an optical sensor when installed in the intraocular location such that deformation can be detected and measured.

2. An optical intraocular sensor system including a sensor according to claim 1 and further comprising: a camera for sensing a characteristic of the deformation structure and a processor for correlating the characteristic to intraocular pressure by image analysis.

3. The sensor system of claim 2, wherein the deformation structure is arranged to deform against the rigid structure and the processor correlates a contact area of the deformation structure against the rigid structure to intraocular pressure.

4. The sensor system of claim 2, wherein the deformation structure is arranged to deform with respect to the rigid structure and the processor correlates a deflection of the deformation structure to intraocular pressure.

5. The sensor system of claim 2, wherein the deformation structure is arranged to deform with respect to the rigid structure and the processor correlates a light intensity pattern to intraocular pressure.

6. The sensor system of claim 2, wherein the deformation structure is arranged to deform with respect to the rigid structure and the processor correlates a light reflection pattern to intraocular pressure.

7. The optical intraocular sensor of claim 1, wherein the deformation structure comprises an elastomer material and the rigid structure comprises rigid layers with the elastomer material between the rigid layers, the sensor further comprising walls sealing to the rigid layers to form an enclosed sensor.

8. The optical intraocular sensor of claim 7, wherein the elastomer material comprises a column of elastomer material.

9. The optical intraocular sensor of claim 7, wherein the elastomer material comprises a layer of elastomer material.

10. The optical intraocular sensor of claim 9, wherein the elastomer material comprises an irregular surface.

11. The optical intraocular sensor of claim 10, wherein the elastomer material comprises a plurality of asperities.

12. The optical intraocular sensor of claim 9, wherein the elastomer material comprises a periodic surface.

13. The optical intraocular sensor of claim 9, wherein the elastomer material comprises a plurality of rounded pillars.

14. The optical intraocular sensor of claim 1, wherein the deformation structure comprises an elastomer membrane and the rigid structure supports the membrane while allowing the membrane to deflect.

15. The optical intraocular sensor of claim 12, wherein the deformation structure further comprises a column that deforms against the rigid structure.

16. The optical intraocular sensor of claim 1, wherein the deformation structure comprises a diaphragm and the rigid structure supports the diaphragm while allowing the diaphragm to deflect.

17. The optical intraocular sensor of claim 16, wherein the diaphragm is suspended by the rigid support structure over a central cavity.

18. The optical intraocular sensor of claim 16, wherein the diaphragm comprises a birefringent material.

19. The optical intraocular sensor of claim 16, wherein the diaphragm comprises a lens.

20. An optical pressure sensor sized to be implanted at an intraocular location and formed from biocompatible materials comprising a rigid structure that supports a deformable structure arranged such that deformation of the deformable structure can be monitored optically when implanted in the intraocular location.

21. A method for sensing intraocular pressure, comprising implanting a sensor at an intraocular location, the sensor comprising a rigid structure that supports a deformable structure, and subjecting the sensor to light stimulation, imaging the deformable structure, and correlating an optical property affected by the state of deformation of the deformable structure to an intraocular pressure.

22. The method of claim 21, wherein said correlating comprises measuring a deformation of the deformable structure.

23. The method of claim 19, wherein said correlating comprises measuring a deflection of the deformable structure.

24. The method of claim 19, wherein said correlating comprises measuring a light reflection pattern.

25. The method of claim 19, wherein said correlating comprises measuring a light intensity pattern.

26. The method of claim 19, wherein said correlating comprises measuring a projection of the deformable structure.
